(19) United States
(12) Patent Application Publication
(10) Pub. No.: US 2006/0292576 A1
(43) Pub. Date: Dec. 28, 2006

(54) Non-in situ hybridization method for detecting chromosomal abnormalities

(75) Inventors: Maher Albitar, Coto de Caza, CA (US); Huai-En Huang Chan, Irvine, CA (US)

Correspondence Address: FOLEY & LARDNER LLP, P.O. BOX 80278, SAN DIEGO, CA, 92138-0278, US

(73) Assignee: Quest Diagnostics Investments Incorporated

(21) Appl. No.: 11/165,445

(22) Filed: Jun. 23, 2005

(51) Int. Cl. C12Q 1/68

(52) U.S. Cl. 435/6

(57) ABSTRACT

The present invention provides methods of detecting chromosomal or genetic abnormalities associated with various diseases or with predisposition to various diseases. In particular, the present invention provides advanced methods of performing DNA hybridization, capture, and detection on solid support. Invention methods are useful for the detection, diagnosis, predicting response to therapy, detecting minimal residual disease, prognosis, or monitoring of disease treatment or progression of particular disease conditions such as cell proliferative disorders

### FIELD OF THE INVENTION

The present invention relates to the use of nucleic acid hybridization complexes comprising target nucleic acid sequences such as DNA or chromosomal fragments and differentially labeled probes in the detection of chromosomal or genetic abnormalities. The invention enables detection of chromosomal or genetic abnormalities without the need for intact cells or partially intact nuclei.

### BACKGROUND OF THE INVENTION

The following description is provided to assist the understanding of the reader. None of the information provided or references cited is admitted to be prior art to the present invention.

Methods of detection of chromosomal abnormalities, such as chromosomal translocations, are well known in the art and include cytogenetic analysis in which a metaphase spread of chromosomes is stained and visualized. Metaphase chromosomes exhibit a particular pattern of light and dark staining manifested in a chromosomal banding pattern. Chromosomal abnormalities (such as aneuploidy, translocations, and deletions, or duplications) can be detected by this technique.

The development of molecular cytogenetic approaches offer assays with greater sensitivity. These techniques incorporate DNA hybridization with a radiolabeled or fluorescent labeled probes. For example, in fluorescence in situ hybridization (FISH) analysis, a fluorescence labeled probe is hybridized to metaphase or interphase chromosomes. Hybridized probe can be detected using a fluorescence microscope.

A number of genetic alterations have been shown to be involved in the development of cancer and other genetic diseases. For example, leukemia is a malignant disease of the blood-forming organs which involves the distorted proliferation and development of leukocytes and their precursors in bone marrow and blood. A particular genetic alteration has been linked with chronic myloid leukemia (CML), a myeloproliferative disorder characterized by increased proliferation of the granulocytic cell line without the loss of the capacity to differentiate. This alteration is an acquired somatic mutation in clonal stem cells, characterized by a reciprocal translocation between chromosomes 9 and 22 resulting in a cytogenetically distinct acrocentric chromosome termed the Philadelphia chromosome. This translocation fuses the BCR gene locus of chromosome 22 and the proto-oncogene ABL locus of chromosome 9 to form a bcr/abl oncogenic protein (Tefferi et al. Mayo Clin Proc 80(3):390-402, 2005). Although the. Philadelphia chromosome was first associated with CML, it is now known to be an indicator of prognosis in other blood disorders such as acute lymphoblastic leukemia (ALL).

Translocations have been linked with other diseases. For example, the fusion of the CBP gene of chromosome 16 to the MLL gene of chromosome 11 through a translocation between chromosomes 11 and 16 has been associated with leukemia (Zhang et al. Genes Chromosomes Cancer 41(3):257-65, 2004). Similarly, a translocation between chromosomes 8 and 21, resulting in a fusion of the AML1 and ETO genes is involved in nearly 15% of acute myeloid leukemia (AML) cases (Zhang et al. Science 305:1286-9, 2004). Further, a number of chromosomal translocations have been identified in various forms of lymphoma. For example, a translocation between chromosomes 8 and 14 involving the c-myc gene is reported to be present in approximately 80-85% of Burkitt lymphoma/leukemia cases (Vega et al. Arch Pathol Lab Med 127:1148-1160, 2003).

Translocations and other genetic abnormalities such as duplications and deletions can be detected through cytogenetic analysis and molecular-based methods (e.g., FISH). However, these methods are all based on intact cells or intact or partially intact nuclei. The present invention provides similar information to FISH but without the need for intact cells or intact nuclei.

### SUMMARY OF THE INVENTION

It is an objective of the present invention to provide improved methods of detecting and analyzing chromosomal abnormalities of interest in a test sample. In preferred embodiments, nucleic acids from a test sample are hybridized to two probes complementary to different segments of a gene of interest or different segments to a chromosomal fragment of interest. One probe is anchored to the solid support while the second probe comprises a detectable label which is used for detection. This method provides for the capture and detection of target nucleic acids hybridizing to both probes simultaneously. Hybridization of both the first and second probes to the same target nucleic acid indicates detection of a chromosomal abnormality in the target nucleic acid, while hybridization of only one of the probes to the same target nucleic acid indicates the absence of a genetic abnormality in the target nucleic acid.

In this and all variants of the invention, the anchored probe may be anchored covalently or non-covalently to the support. If non-covalent attachment is used, a preferred method is via a “binding pair,” which refers herein to two molecules which form a complex through a specific interaction. Thus, the nucleic acid probe can be captured on the solid support through an interaction between one member of the binding pair linked to the probe and the other member of the binding pair coupled to the solid support. A binding pair member also can be used to link the detectable label to the other nucleic acid probe. In a preferred embodiment, the binding pair is biotin and avidin or streptavidin. In other embodiments the binding pair is comprised of a ligand-receptor, a hormone-receptor, an antigen-antibody, or an oligonucleotide-complement.

In some variants of the method, the two probes may be hybridized to the target nucleic acid in a liquid and then the complex can be captured by a solid support. The anchored probe in this approach is preferably anchored non-covalently and preferably via a binding pair. In other variants, the solid support may first comprise the anchored probe, which is then contacted for hybridization with the target nucleic acid, alone or together with the labeled probe.

The information available from methods disclosed herein is similar to what can be obtained by FISH but without the need for intact cells or intact nuclei. Where FISH involves hybridization to intact chromosomes in a metaphase spread, hybridization in the present invention can be conducted in a liquid phase. Although not wishing to be limited by this term, the present invention for ease of understanding may be viewed as a liquid hybridization form of FISH, i.e. “liquid FISH.”

According to one aspect of the present invention, there are provided methods of detecting the presence or absence of a genetic abnormality in a target nucleic acid in a test sample. The method includes forming on a solid support a complex comprising the target nucleic acid, a first nucleic acid probe hybridizing to a first segment of the target nucleic acid, the first nucleic acid probe labeled with a detectable label, and a second nucleic acid probe hybridizing to a second segment of the target nucleic acid, the second nucleic acid probe anchored to the solid support. The complex is detected by detecting incorporated detectable label, wherein hybridization of both the first and second probes to the same target nucleic acid indicates the presence of genetic abnormality in the target nucleic acid, while hybridization of only one of the probes to the same target nucleic acid indicates the absence of a genetic abnormality in the target nucleic acid.

According to another aspect of the present invention, there are provided methods of detecting a chromosomal translocation in the nucleic acid of a test sample. The method includes the hybridization of two nucleic acid probes, one complementary to a sequence of the donor chromosome segment and the other complementary to a sequence of the recipient chromosome which adjoins or is near to the inserted donor chromosome segment. One probe is anchored to the support and the other probe is labeled with a detectable label. A test sample of genomic DNA hybridizing to both probes will form a complex on the support or such a complex is preformed and then captured on a solid support and detected via the detectable label. The quantity of captured, labeled complex from the test sample represents the test value. If the test value shows that label is associated with captured hybridization complexes, the test sample is determined to contain the chromosomal translocation. In one embodiment, one can compare the test value for the test sample with a test value from a reference sample which contains the target gene but lacking the translocation.

According to another aspect of the present invention, there are provided methods of detecting a duplication or deletion in a particular target chromosomal region or gene in an individual. The method includes forming on a solid support a complex comprising the nucleic acid associated with the particular chromosomal region or gene which is obtained from the sample, a labeled nucleic acid probe hybridizing to a first segment of the particular chromosomal region or gene, and a second nucleic acid probe hybridizing to a second segment of the particular chromosomal region or gene, wherein the second nucleic acid probe is anchored to the solid support. In a preferred embodiment, the target nucleic acid is genomic DNA which has been fragmented. The quantity of captured, labeled complex from the test sample represents the test value. The test value may be compared to a control value which may be obtained from the quantity of complex obtained from a different target gene or chromosomal region preferably from the same sample. A higher test value as compared to the control value is indicative of duplication or amplification, whereas a lower test value as compared to control value is indicative of a chromosomal or gene deletion. In another approach, one can determine a ratio of the test value of the test sample to the control value in that sample and compare to a similar ratio representing the test value and control value of a reference sample which contains nucleic acid that does not contain a deletion, duplication, or amplification in the chromosomal region or gene of interest.

According to another aspect of the present invention, there are provided methods of determining the diagnosis, predicting response to therapy, detecting minimal residual disease or prognosis of a disease in an individual. In this method, a complex is formed between a target nucleic acid from a test sample, a probe comprising a detectable label and hybridizing to one segment of a target nucleic acid and a second probe anchored to the support and hybridizing to a second segment of the target nucleic acid. The amount of complex on the solid support is measured through detection of incorporated detectable label of the first probe. The amount of complex formed is compared to the amount of complex formed in a similar manner from a sample obtained from a reference sample. The reference sample may be obtained from a normal individual, wherein a difference between the measurements from the test and reference samples is correlated with diagnosis or prognosis of a disease.

According to another aspect of the present invention, there are provided methods of monitoring treatment or progression of a disease. In this method samples are obtained from a patient at different points in time (e.g., before and after a regimen of treatment of the disease). A complex is formed between a target nucleic acid from the first sample, a probe comprising a detectable label and hybridizing to one segment of a target nucleic acid and a second probe anchored to the support and hybridizing to a second segment of a target nucleic acid. The amount of complex on the support from the first sample is compared to the amount of complex formed using the same probes and target nucleic acid from the second sample. A difference in amount of complex formed can be correlated to progression of the disease or success of the treatment regimen.

According to another aspect of the present invention, there are provided methods of measuring tumor burden in an individual. In this method, a complex is formed on a solid support between a target nucleic acid from a test sample, a probe comprising a detectable label and hybridizing to one segment of a target nucleic acid and a second probe anchored to the support and hybridizing to a second segment of the target nucleic acid. The amount of complex on the solid support is measured through detection of incorporated detectable label of the first probe. The amount of complex formed is compared to a reference value or set of values of the amount of complex formed in a similar manner from a sample obtained from a reference sample, from a patient whose tumor burden is known, to determine tumor burden of the test sample.

As used herein the term “tumor burden” refers to the amount in volume or mass of tumor in an individual. This amount may be at one site, such as the primary tumor, or may be the amount in aggregate from multiple sites such as the primary and/or metastases.

In another embodiment, methods of determining tumor burden include the formation of two complexes on solid support. The first complex comprises a first target nucleic acid from a test sample from the individual and two nucleic acid probes; one containing a detectable label and the other anchored to the support. The second complex comprises a second or control target nucleic acid from the test sample and two different nucleic acid probes, one containing a detectable label, distinguishable from the label of the first complex, and the other probe anchored to the solid support. The amount of each of the two complexes is measured and a test ratio determined. This ratio is then compared to a reference ratio or set of ratios that correlate the test ratio to tumor burden.

According to another aspect of the present invention, there is provided a method of diagnosing CML by detecting the Philadelphia chromosome, characterized by a specific reciprocal translocation between the BCR locus of chromosome 22 and the ABL locus of chromosome 9. The method includes the hybridization of two nucleic acid probes, one containing the BCR locus and the other containing the ABL locus, with a sample of restriction endonuclease digested genomic DNA. The first probe is labeled with biotin and the second probe is detectably labeled. The probes are combined with a test sample of genomic DNA under hybridizing conditions. The hybridization product is then captured on a solid support (e.g., beads or microparticles) through a specific interaction between streptavidin or avidin on the beads and biotin on the first probe. When the test sample genomic DNA contains a translocation between BCR and ABL, the nucleic acid will hybridize to both probes forming a complex that can be captured on the beads. The beads can then be run through a flow cytometer and the detectable label on the second probe can be measured. Detection of the label indicates that the test sample genomic DNA contains the BCR-ABL translocation.

As readily recognized by those of skill in the art, an assay to detect any known chromosomal translocation can be devised through construction of nucleic acid probes comprising the portions of the chromosomes known to be involved in the translocations. These probes can be synthetic or derived from a BAC or other artificial chromosome containing the chromosomes of interest. Examples of other translocations that may be detected are t(11;16)—the fusion of the CBP gene of chromosome 16 to the MLL gene of chromosome, t(8;21)−the fusion of the AML1 and ETO genes, and t(8;14) involving the c-myc gene, t(14, 18) involves BCL2, t(11;14) involves BCL1, inv 16 involves core binding protein, and t(4;14), or t(5;12).

In any of these methods, the test and control values may be assayed simultaneously using variations in the solid support (e.g., different size beads) and/or different labels for the second probe (e.g., distinguishable fluorescent dyes). Also, the test and reference nucleic acid may be obtained from any number of sources and methods. For example, the test sample can be DNA extracted from viable cells, free circulating DNA in body fluids (plasma, serum, urine, central system fluid, stool, bile duct, paraffin-embedded tissue, and the like). In any of the methods of the invention, two or more adjacent probes may be used as the labeled probe to increase the signal of the detection.

As used herein, “nucleic acid” refers broadly to segments of a chromosome, segments or portions of DNA, cDNA, and/or RNA. Nucleic acid may be derived or obtained from an originally isolated nucleic acid sample from any source (e.g., isolated from, purified from, amplified from, cloned from, reverse transcribed from sample DNA or RNA).

“Target nucleic acid” as used herein refers to segments of a chromosome, a complete gene with or without intergenic sequence, segments or portions a gene with or without intergenic sequence, or sequence of nucleic acids to which probes are designed. Target nucleic acids may may include wild type sequences, nucleic acid sequences containing mutations, deletions or duplications, or any other gene of interest. Target nucleic acids may represent alternative sequences or alleles of a particular gene. Target nucleic acids may be derived from genomic DNA, cDNA, or RNA. As used herein target nucleic acid is preferably native DNA and not a PCR amplified product. Target nucleic acid can be large fragments of DNA, about 20 kb or more.

“Genomic nucleic acid” or “genomic DNA” refers to some or all of the DNA from the nucleus of a cell directly or indirectly isolated or derived in some manner therefrom. Genomic DNA may be intact or fragmented (e.g., digested with restriction endonucleases by methods known in the art). In some embodiments, genomic DNA may include sequence from all or a portion of a single gene or from multiple genes, sequence from one or more chromosomes, or sequence from all chromosomes of a cell. In contrast, the term “total genomic nucleic acid” is used herein to refer to the full complement of DNA contained in the genome of a cell. As is well known, genomic nucleic acid includes gene coding regions, introns, 5′ and 3′ untranslated regions, 5′ and 3′ flanking DNA and structural segments such as telomeric and centromeric DNA, replication origins, and intergenic DNA. Genomic nucleic acid may be obtained from the nucleus of a cell, or recombinantly produced. Genomic DNA also may be transcribed from DNA or RNA isolated directly from a cell nucleus. PCR amplification also may be used. Methods of purifying DNA and/or RNA from a variety of samples are well-known in the art.

The terms “allele” and “allelic variant” are used interchangeably herein. An allele is any one of a number of alternative forms or sequences of the same gene occupying a given locus or position on a chromosome. A single allele for each locus is inherited separately from each parent, resulting in two alleles for each gene. An individual having two copies of the same allele of a particular gene is homozygous at that locus whereas an individual having two different alleles of a particular gene is heterozygous.

The term “diagnose” or “diagnosis” as used herein refers to the act or process of identifying or determining a disease or condition in a mammal or the cause of a disease or condition by the evaluation of the signs and symptoms of the disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease.

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of years, on average, that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

A prognosis is often determined by examining one or more prognostic factors or indicators. These are markers, such as the presence of a particular chromosomal translocation, the presence or amount of which in a patient (or a sample obtained from the patient) signal a probability that a given course or outcome will occur. The skilled artisan will understand that associating a prognostic indicator with a predisposition to an adverse outcome may involve statistical analysis.

As used herein, “chromosomal abnormality” refers to any difference in the DNA sequence from a wild-type or normal or a change in chromosomal copy number. A chromosomal abnormality may reflect a difference between the full genetic complement of all chromosomes contained in an organism, or any portion thereof, as compared to a normal full genetic complement of all chromosome in that organism. For example, a chromosomal abnormality may include a change in chromosomal copy number (e.g., aneuploidy), or a portion thereof (e.g., deletions, duplications, amplifications); or a change in chromosomal structure (e.g., translocations, mutations). “Aneuploid cell” or “aneuploidy” as used herein, refers to a cell having an abnormal number of at least one chromosome in interphase. A chromosome “translocation” is the interchange of parts between nonhomologous chromosomes. It is generally detected through cytogenetics or a karyotyping of affected cells. There are two main types, reciprocal, in which all of the chromosomal material is retained and Robertsonian, in which some of the chromosomal material is lost. Further, translocations can be balanced (in an even exchange of material with no genetic information extra or missing) or unbalanced (where the exchange of chromosome material is unequal resulting in extra or missing genes).

Chromosomal abnormalities that can be detected by the method of the invention include deletions, duplications, amplifications and translocations, and the like. The method is particularly suitable for large abnormalities such as involving at least 50 bp, more preferably at least 100 bp, more preferably at least 200 bp, more preferably at least 500 bp, more preferably at least 1 kb, more preferably at least 2 kb, more preferably at least 4 kb, more preferably at least 8 kb, and even more preferably at least 10 kb. However, smaller abnormalities may be detected including at least 5 bp, at least 10 bp, and at least 25 bp by appropriate adjustment of probes and hybridization conditions as is well known in the art.

As used herein, “genetic abnormality” refers to a chromosomal abnormality that is known to be associated with a particular disease condition (e.g., a specific gene mutation causing a dysfunctional protein directly causing a disease state). A chromosomal or genetic abnormality may be hereditary, i.e., passed from generation to generation.

A “sample” as used herein may be acquired from essentially any diseased or healthy organism, including humans, animals and plants, as well as cell cultures, recombinant cells, cell components and environmental sources. Samples may be from any animal, including by way of example and not limitation, humans, dogs, cats, sheep, cattle, and pigs. Samples can be a biological tissue, fluid or specimen. Samples may include, but are not limited to, amniotic fluid, blood, blood cells, cerebrospinal fluid, fine needle biopsy samples, peritoneal fluid, plasma, pleural fluid, saliva, semen, serum, sputum, tissue or tissue homogenates, tissue culture media, urine, and the like. Samples may also be processed, such as sectioning of tissues, fractionation, purification, or cellular organelle separation.

A “test sample” comprises nucleic acids or other nucleic acids typically from a patient or cell population suspected of, or being screened for, having one or more cell or DNA containing a chromosomal or genetic abnormality. A test sample may comprise genomic DNA or mRNA from which cDNA can be made. A test sample can contain or be used as a source of target nucleic acids for the methods of the invention. A test sample may contain nucleic acid that has not been amplified.

A “reference sample” comprises target nucleic acids typically from a normal patient or wild-type cell population with a normal genetic profile. In other embodiments, a reference sample may be taken from a patient with a known disease or disorder. The reference sample may comprise genomic DNA or mRNA from which cDNA can be made. A reference sample can contain or be used as a source of target nucleic acids for the methods of the invention. A test sample may contain nucleic acid that has not been amplified.

A “reference” or “reference nucleic acid” may be a target nucleic acid containing a housekeeping gene or locus or other gene that is not expected to change under varying conditions (e.g., a normal state or a disease state). A reference may also represent a gene in a normal or wild type state, that is, absent mutations, translocations, deletions, or duplications.

A “test value” is obtained through a determination of the amount of complex formed from the nucleic acids of a test sample comprising the target nucleic acid sequence where the target for hybridization is suspected of having a genetic abnormality. A test value also can be obtained by detecting the same chromosomal or gene sequence in a reference sample.

A “control value” is obtained through a determination of the amount of complex formed from the nucleic acids of a test or reference sample where the target nucleic acid sequence that is being detected is not one that is associated with a genetic abnormality.

A “reference value” refers to a value that has been related to some other characteristic. A set of reference values can be used as a standard curve.

The test value or control value may be expressed as an “amount” or copy number of complex. An amount complex can be a single value or a range of values corresponding to the level of detection of incorporated label (e.g., fluorescence intensity). For example, a range of values may be used to generate a standard curve relationship between the amount of complex formed versus some other quantity (e.g., tumor burden).

The test value or control value may be expressed as a “relative amount” or “ratio” of the amount of one complex to the amount of another. In certain embodiments of the invention methods, the two complexes may be obtained using the same target gene, wherein the amount of the second complex represents a historical value or a value obtained in a parallel assay. In other embodiments, the two complexes are obtained using two different genes, the first being a gene of interest and the second being a gene not expected to change (e.g., a housekeeping gene). Relative amounts may be a single value or a range of values. For example, a range of values may be used to generate a standard curve relationship between the relative amount of complex formed versus some other quantity (e.g., tumor burden).

The nucleic acids from the test sample and nucleic acid probes are contacted under hybridization conditions. The term “hybridization” as used herein, refers to the pairing of substantially complementary nucleotide sequences (strands of nucleic acid) to form a duplex or heteroduplex through formation of hydrogen bonds between complementary base pairs. It is a specific, i.e., non-random, interaction between two complementary polynucleotides. Hybridization and the strength of hybridization (i.e., the strength of the association between the nucleic acids) is influenced by such factors as the degree of complementary between the nucleic acids, stringency of the conditions involved, and the Tₘ of the formed hybrid.

Nucleic acid probes may be produced synthetically by methods known in the art or may be derived by copy of cloned or genomic DNA or RNA or by fragmentation of genomic DNA or artificial chromosomes. Nucleic acid probes useful in the methods of the invention are preferably at least 50 nucleotides in length, more preferably at least 100, at least 500, at least 1000, at least 2,000, at least 5,000, at least 10,000, at least 20,000, nucleotides, at least 40,000, at least 80,000, at least 120,000, nucleotides nucleotides length. Generally, probes of 70,000 to 100,000 nucleotides in length are preferred. The longer probes may be derived from intact artificial chromosomes containing nucleic acid segment of interest is between about 1,000 (1 kb) and about 1,000,000 (1 Mb) nucleotides in length. Nucleic acid probes useful in the methods of the invention are preferably large fragments of DNA (>20 kb, including cosmid, yac, or BAC clones) in a fashion similar to that used in cellular-based FISH.

The term “label” as used herein, refers to any molecule directly associated with a nucleic acids of a sample such that substantially all individual nucleic acid segments of that sample can be detected or captured via the same label. The label may be a detectable label or part of a binding pair.

Nucleic acid probes may be directly detectable via linkage to a detectable label. A “detectable label” as used herein refers any moiety used to achieve a hybridization signal detectable by spectroscopic, photochemical, biochemical, immunochemical, electromagnetic, radiochemical, or chemical means, such as fluorescence, chemifluoresence, or chemiluminescence, or any other appropriate means. Preferred detectable labels include fluorescent dye molecules, or fluorophores, such as fluorescein, phycoerythrin, Cy3™, Cy5™, allophycocyanine, Texas Red, peridenin chlorophyll, cyanine, FAM, JOE, TAMRA, tandem conjugates such as phycoerythrin-Cy5™, and the like. The detectable label may be linked directly or indirectly to the samples of nucleic acids prior to or after hybridization.

The phrase “binding pair” as used herein refers to two molecules which form a complex through a specific interaction. As used herein, one of the members of the binding pair comprises a label linked to one of the nucleic acid probes. The second member of the binding pair is coupled to the solid support. The nucleic acid probe can be captured on the solid support through an interaction member of the binding pair linked to the probe and the member of the binding pair coupled to the solid support. In this way, the nucleic acid probe linked to the label and any nucleic acids hybridized thereto can be captured on solid support. In a preferred embodiment, the binding pair is biotin and avidin or streptavidin. In other embodiments the binding pair is comprised of a ligand-receptor, a hormone-receptor, an antigen-antibody, or an oligonucleotide-complement.

A binding pair may be used as indirect detectable labels. For example, a nucleic acid probe is linked to a first member of a binding pair and the second member of a binding pair is linked to a detectable label. The nucleic acid probe can then be detected via the interaction of the members of the binding pair.

The phrases “solid support” and “solid support” are used interchangeably herein and refer to beads, microparticles, microspheres, plates which are flat or comprise wells or shallow depressions or grooves, microwell surfaces, slides, chromatography columns, membranes, filters, microchips, and the like, which capture hybridization complexes through a specific interaction between two members of a binding pair. In preferred embodiments beads or microparticles are substantially the same size. In other embodiments, beads or microparticles are of one or more sizes. Beads or microparticles may be magnetic or not.

### BRIEF DESCRIPTION OF THE FIGURES

FIG. 1 illustrates an exemplary embodiment of the present invention for detecting a translocation between chromosomes 9 and 22 generating b2a2 p210 BCR/Abl fusion protein. A probe for the BCR segment is labeled with biotin as indicated while a second probe for ABL is labeled with a detectable label (“digoxigenin”). The probes are hybridized to digested genomic DNA and the complexes captured on a streptavidin coated beads. Binding of digoxigenin labeled probe to the beads is measured by detecting fluorescence associated with a flurosecent labeled anti-digoxigenin antibody. The normal target nucleic acid is shown attached to the bead via the hybridized BCR probe but not hybridized to the ABL probe, while the translocated target is shown attached to the bead via the BCR probe and also hybridized to the labeled ABL probe.

FIG. 2 is a chromatogram of detection of nucleic acid with a 9/22 translocation by flow cytometry of hybridization complexes captured on streptavidin coated beads as described in FIG. 1.

FIG. 3 illustrates an exemplary embodiment of the present invention for quantitating the relative ratio of a translocation between chromosomes 9 and 22 generating b2a2 p210 BCR/Abl fusion protein. A probe for the BCR segment is labeled with biotin as indicated while a second probe for ABL is labeled with a detectable label (“digoxigenin”). A third probe hybridizing downstream of BCR in a segment of the chromosome that is deleted by the 9/22 translocation is labeled with FITC. The probes are hybridized to digested genomic DNA and the complexes captured on a streptavidin coated beads. Binding of digoxigenin labeled probe to the beads is measured by detecting fluorescence associated with a flurosecent labeled (phycoerythrin) anti-digoxigenin antibody while binding of FITC labeled probe to beads is measured by detected by detecting fluorescence associated with a flurosecent labeled (alexa fluor 488) anti-FITC antibody. The relative ratio of digoxigenin to FITC probe binding is determined.

FIG. 4 illustrates an exemplary embodiment of the present invention for detecting a deletion in chromosome 5. The deleted allele is shown in the upper drawing with a probe for a gene segment of chromosome 5 labeled with biotin and a second probe hybridizing downstream to a segment that is deleted from this allele, the second probe labeled with a detectable label (“digoxigenin”). In the same nucleic acid sample, a control non-deleted reference allele on both versions of chromosome 21 (bottom two drawings) is evaluated using an upstream probe labeled with biotin and a downstream probe labeled with FITC. The probes are hybridized to digested genomic DNA and the complexes captured on streptavidin coated beads. Binding of digoxigenin labeled probe to the beads is measured by detecting fluorescence associated with a flurosecent labeled (phycoerythrin) anti-digoxigenin antibody while binding of FITC labeled probe to beads is measured by detected by detecting fluorescence associated with a flurosecent labeled (alexa fluor 488) anti-FITC antibody. The relative ratio of digoxigenin to FITC probe binding is determined.

### DETAILED DESCRIPTION OF THE INVENTION

In accordance with the present invention, there are provided methods of detecting a target nucleic acid through hybridization of nucleic acid from a test sample with two nucleic acid probes, one probe providing for detection of the hybridization complex and the other providing for capture of the hybridization complex on a solid support. In this formulation of the invention, at least one of the probes is 50 nucleotides in length. Hybridization of both probes to the target nucleic acid is required for capture and detection of the complex. This method is especially useful in the detection of chromosomal or genetic abnormalities such as translocation, deletion, and duplication.

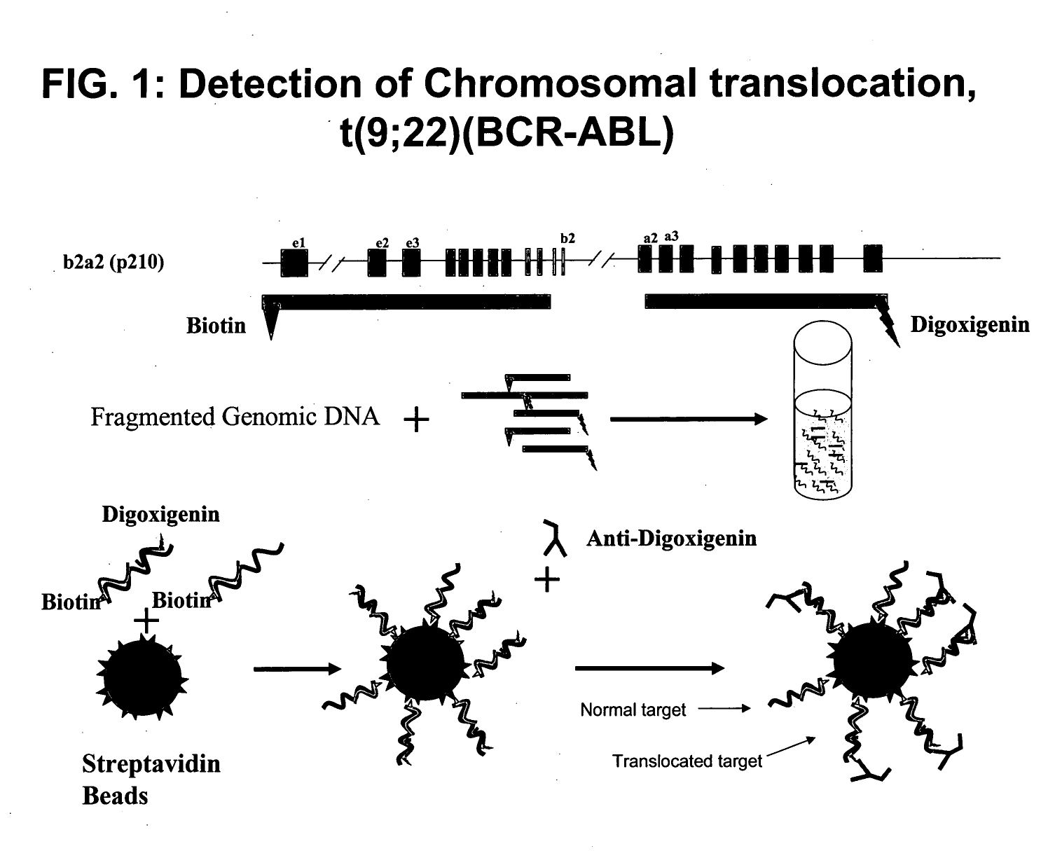

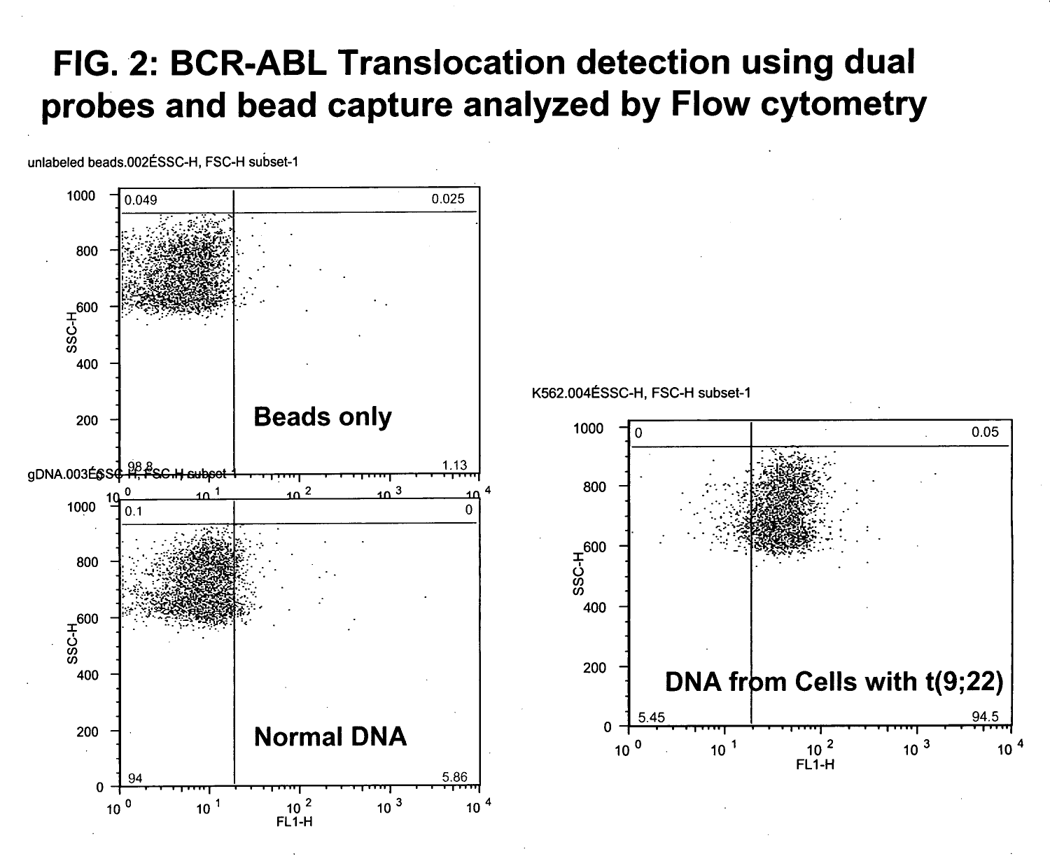

The method for detecting a translocation exemplified as the BCR/ABL translocation resulting in the Philadelphia chromosome is shown schematically in FIG. 1. Flow cytometry can be used to detect the hybridization complex captured on beads as shown in FIG. 2. Beads only and normal DNA are used as controls. Increased amounts of fluorescence is detected on beads when nucleic acid containing the BCR/ABL tanslocation is hybridized to the two labeled probes.

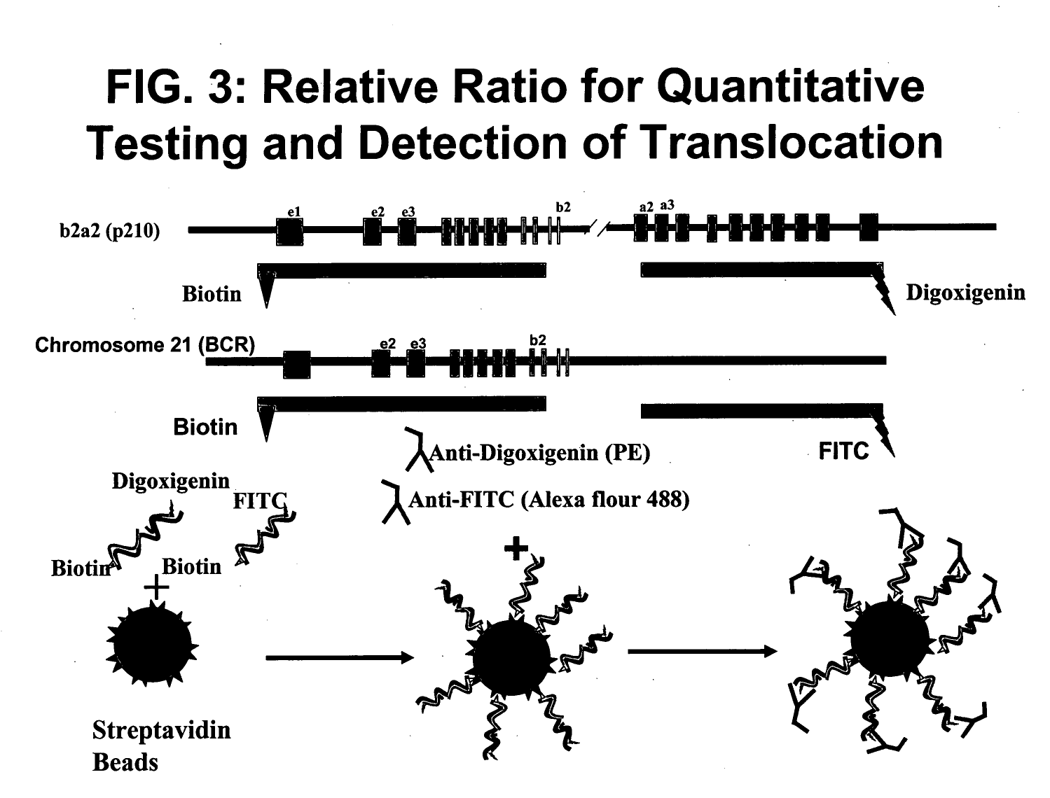

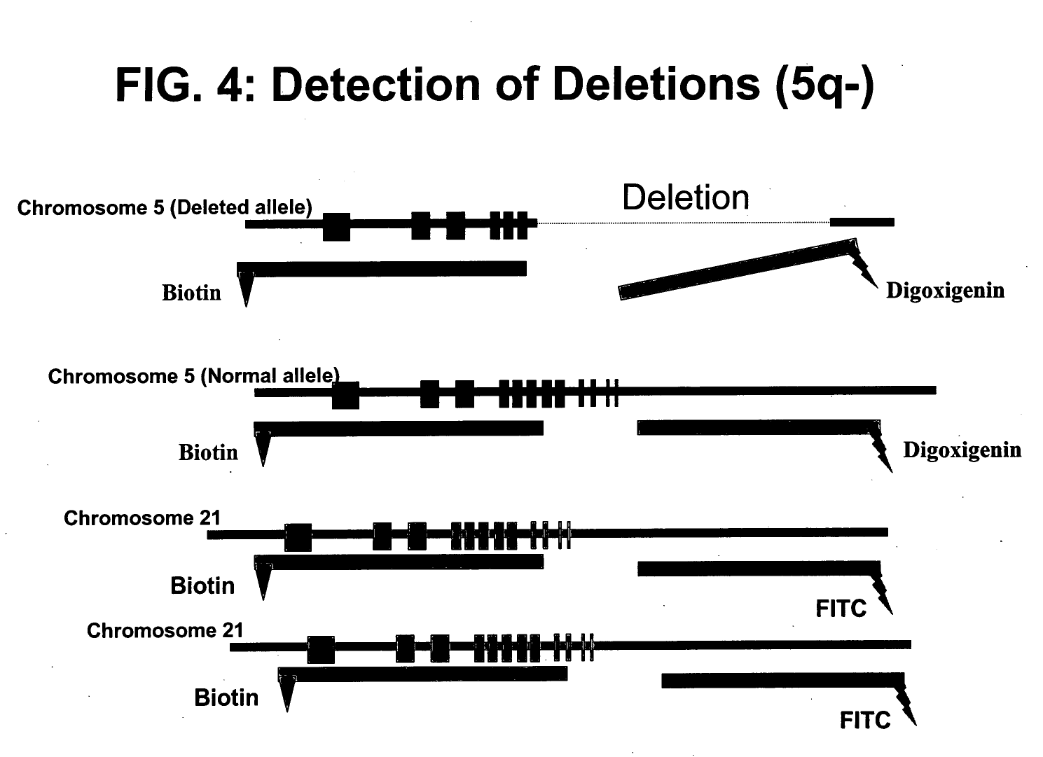

One can obtain quantitative data about the extent of translocated DNA to normal DNA in a sample by the approach shown in FIG. 3 with the BCR/ABL shown in the example. In this case, in addition to the probe set for detecting the translocation such as shown in FIG. 1, a third probe is used to detect the wildtype allele. Thus, in example in FIG. 3, the third probe hybridizes downstream of BCR in a segment of the chromosome that is present in the wildtype, but deleted by the 9/22 translocation. This third probe is differentially labeled, in this case with FITC, so it can be distinguished from the probe used to detect the translocation. The probes and target nucleic acid are hybridized and the complexes captured on streptavidin coated beads. The amount of probe binding for the translocation is measured and compared to the amount of probe binding for the wildtype allele. In FIG. 4, digoxigenin labeled probe binding to the translocation allele is measured by detecting fluorescence associated with a flurosecent labeled (phycoerythrin) anti-digoxigenin antibody while the amount of wildtype allele detected binding with the FITC labeled probe is detected by detecting fluorescence associated with a flurosecent labeled (alexa fluor 488) anti-FITC antibody. The level of staining on the beads is determined by evaluating the percentage of beads positive and median intensity of positivity for these beads. To encompass both parameters, the concept of INDEX is used.
The Index (molecule/100 beads)=(% of positive beads)×(median intensity)
The relative ratio of digoxigenin to FITC probe binding indicates the relative amount of translocation containing DNA versus wildtype DNA in the sample.

One can determine from the percent of binding of the mutant form of the DNA versus the wildtype form of the DNA, the percentage of cells in a sample from the individual with the mutant form of the DNA. This can be done with the following formula.
Actual %=(200 X)/(X+Y)

X=number of copies of the test locus of the chromosome

Y=number of copies of the control locus of chromosome

For example, assuming that there are 100 cells in the samples; and each cell has 2 copies (chromosomes); and ABL is the internal control, and a sample where the 20% of the cells carry a fused BCR-ABL translocated allele, the sample will have 20 copies showing fusion BCR-ABL and 180 copies showing normal ABL.
The formula: 200(20)/(200)=20% of cells carry the translocation.

This formula assumes one flourochrome per molecule of DNA/antibody. Since each molecule represents one allele and since the internal control is used for controlling for the amount of DNA in the sample, one can measure the relative number of cells (%) carrying the abnormality in the test sample.

If we use an independent locus (or gene) as the control value to quantify the percentage of cells with a deletion on a different chromosome, the formula is:
Actual %=2(Y−X)

Assuming:

X=number of copies of the test locus of the chromosome

Y=number of copies of the control locus of chromosome

This formula assumes one flourochrome per molecule of DNA/antibody. Since each molecule represents one allele and since the internal control is used for controlling for the amount of DNA in the sample, one can measure the relative number of cells (%) carrying the abnormality in the test sample.

In a related manner, FIG. 4 illustrates how one determines if an individual has a deletion, duplication or amplification of a particular gene or chromosomal segment. One probe which hybridizes near to the deletion site of both the mutant and wildtype forms of nucleic acid and the a second probe hybridizes to the segment that is deleted. As shown in FIG. 4, the upstream probe is labeled with biotin and the downstream probe hybridizing to the segment that is deleted from this allele is labeled with a detectable label (“digoxigenin”). If the test nucleic acid contains the deletion, the amount of signal in this example would be lower than normal since the there would be less binding of the second probe. To control for variations in the assay, a second hybridization is done simultaneously or in parallel to determine the extent of hybridization to a reference gene or genomic segment. The bottom two drawings in FIG. 4 depict the reference hybridization showing detectable labeled probe binding for both chromosomes. The Example shown in FIG. 4 depicts simultaneous detection of the test and reference targets in the same assay with a single sample of nucleic acid. In this case, binding of digoxigenin labeled probe to the beads is measured by detecting fluorescence associated with a flurosecent labeled (phycoerythrin) anti-digoxigenin antibody while binding of FITC labeled probe to beads is measured by detected by detecting fluorescence associated with a flurosecent labeled (alexa fluor 488) anti-FITC antibody. The relative ratio of digoxigenin to FITC probe binding is determined. This is then compared to the ratio for the nucleic acid that is wildtype for both the test and reference genes or chromosomal segment under evaluation. Increases over the control ratio indicate duplication or amplification while decreases relative to the control ratio indicate deletion.

Sources of Nucleic Acids

The methods of the present invention can be used to detect a chromosomal abnormality in a test sample. Methods of obtaining test samples are well known to those of skill in the art and include, but are not limited to, aspirations, tissue sections, drawing of blood or other fluids, surgical or needle biopsies, and the like. The test sample may be obtained from an individual or a patient who is suspected of having a genetic abnormality. The test sample may contain cells, tissues or fluid obtained from a patient suspected of having a pathology or a condition associated with a chromosomal or genetic abnormality. The test sample may be liquid without any cells or tissue. Samples may include, but are not limited to, amniotic fluid, biopsies, blood, blood cells, bone marrow, cerebrospinal fluid, fecal samples, fine needle biopsy samples, peritoneal fluid, plasma, pleural fluid, saliva, semen, serum, sputum, tears, tissue or tissue homogenates, frozen or paraffin sections of tissue, tissue culture media, cells or cell lysates from culture, and urine. Samples may also be processed, such as sectioning of tissues, fractionation, purification, or cellular organelle separation.

The invention methods can be used to perform prenatal diagnosis using any type of embryonic or fetal cell or nucleic acid containing body fluid. Fetal cells can be obtained through the pregnant female, or from a sample of an embryo. Thus, fetal cells are present in amniotic fluid obtained by amniocentesis, chorionic villi aspirated by syringe, percutaneous umbilical blood, a fetal skin biopsy, a blastomere from a four-cell to eight-cell stage embryo (pre-implantation), or a trophectoderm sample from a blastocyst (pre-implantation or by uterine lavage).

In particular embodiments, genomic DNA may be used. Genomic DNA may be isolated from cells or tissues using standard methods, see, e.g., Sambrook, et al., 1989, Molecular Cloning: A Laboratory Manual, Second Edition, Cold Spring Harbor Press, Plainview, N.Y.

In other embodiments, mRNA or cDNA generated from mRNA or total RNA may be used. RNA is isolated from cells or tissue samples using standard techniques, see, e.g., Sambrook, et al., 1989, Molecular Cloning: A Laboratory Manual, Second Edition, Cold Spring Harbor Press, Plainview, N.Y. In addition kits for isolating mRNA and synthesizing cDNA are commercially available.

Solid Supports

Solid supports may be beads, microparticles, microspheres, plates which are flat or comprise wells or shallow depressions or grooves, microwell surfaces, slides, chromatography columns, membranes, filters, microchips, and the like, which capture hybridization complexes through a specific interaction between two members of a binding pair. The bound probe may anchored in an fixed array or matrix on the support such as in the case of a flat or relatively flat surface or in pits or wells arrayed on a surface (e.g., a microwell plate). Alternatively, the surface may be individualized for each assay where no array is used. For example, the solid support may be beads which are not arranged in an array and are read individually by a cell sorter.

Nucleic Acid Probes

Nucleic acid probes may be generated synthetically by methods known in the art or may be derived by enzymatic DNA synthesis or amplification of cloned or genomic DNA or RNA or by fragmentation of genomic DNA or artificial chromosomes.

In a preferred embodiment, the nucleic acid probes are derived from one, several or all of the human genomic nucleic acid segments provided in a compendium of bacterial artificial chromosomes (BACs) compiled by The BAC Resource Consortium. These probes are usually referred to in the art by their RPI or CTB clone names, see Cheung et al., Nature 409:953-958, 2001. This compendium contains 7,600 cytogenetically defined landmarks on the draft sequence of the human genome (see McPherson et al., Nature 409:934-41, 2001). These landmarks are large-insert clones mapped to chromosome bands by fluorescence in situ hybridization, each containing a sequence tag that is positioned on the genomic sequence. These clones represent all 24 human chromosomes in about 1 Mb resolution. Sources of BAC genomic collections include the BACPAC Resources Center (CHORI—Children's Hospital Oakland Research Institute), ResGen (Research Genetics through Invitrogen) and The Sanger Center (UK).

Association of Label with Nucleic Acid probes

Useful labels include, e.g., fluorescent dyes (e.g., Cy5™, Cy3™, FITC, rhodamine, lanthamide phosphors, Texas red), ³²P, ³⁵S, ³H, ¹⁴C, ¹²⁵I, ¹³¹I, electron-dense reagents (e.g., gold), enzymes, e.g., as commonly used in an ELISA (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can be directly incorporated into the nucleic acid to be detected, or it can be attached to a probe (e.g., an oligonucleotide) or antibody that hybridizes or binds to the nucleic acid to be detected.

In preferred embodiment the detectable label is a fluorophore. The term “fluorophore” as used herein refers to a molecule that absorbs light at a particular wavelength (excitation frequency), and subsequently emits light of a different, typically longer, wavelength (emission frequency) in response. Suitable fluorescent moieties include the following fluorophores known in the art:
4-acetamido-4′-isothiocyanatostilbene-2,2′disulfonic acid
acridine and derivatives:
   acridine
   acridine isothiocyanate
Alexa Fluor® 350, Alexa Fluor® 488, Alexa Fluor® 546, Alexa Fluor® 555, Alexa Fluor® 568,
Alexa Fluor® 594, Alexa Fluor® 647 (Molecular Probes)
5-(2′-aminoethyl)aminonaphthalene-1-sulfonic acid (EDANS)
4-amino-N-[3-vinylsulfonyl)phenyl]naphthalimide-3,5 disulfonate (Lucifer Yellow VS)
N-(4-anilino-1-naphthyl)maleimide
anthranilamide
Black Hole Quencher™ (BHQ™) dyes (biosearch Technologies)
BODIPY® R-6G, BOPIPY® 530/550, BODIPY® FL
Brilliant Yellow
coumarin and derivatives:
   coumarin
   7-amino-4-methylcoumarin (AMC, Coumarin 120)
7-amino-4-trifluoromethylcouluarin (Coumarin 151)
Cy2®, Cy3®, Cy3.5®, Cy5®, Cy5.5®
cyanosine
4′,6-diaminidino-2-phenylindole (DAPI)
5′,5″-dibromopyrogallol-sulfonephthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansyl chloride)
4-(4′-dimethylaminophenylazo)benzoic acid (DABCYL)
4-dimethylaminophenylazophenyl-4′-isothiocyanate (DABITC)
Eclipse™ (Epoch Biosciences Inc.)
eosin and derivatives:
   eosin
   eosin isothiocyanate
erythrosin and derivatives:
   erythrosin B
   erythrosin isothiocyanate
ethidium
fluorescein and derivatives:
   5-carboxyfluorescein (FAM)
   5-(4,6-dichlorotriazin-2-yl)aminofluorescein (DTAF)
   2′,7′-dimethoxy-4′5′-dichloro-6-carboxyfluorescein (JOE)
   fluorescein
   fluorescein isothiocyanate (FITC)
   hexachloro-6-carboxyfluorescein (HEX)
   QFITC (XRITC)
   tetrachlorofluorescein (TET)
fluorescamine
IR144
IR1446
Malachite Green isothiocyanate
4-methylumbelliferone
ortho cresolphthalein
nitrotyrosine
pararosaniline
Phenol Red
B-phycoerythrin, R-phycoerythrin
o-phthaldialdehyde
Oregon Green®
propidium iodide
pyrene and derivatives:
   pyrene
   pyrene butyrate
   succinimidyl 1-pyrene butyrate
QSY® 7, QSY® 9, QSY® 21, QSY® 35 (Molecular Probes)
Reactive Red 4 (Cibacron® Brilliant Red 3B-A)
rhodamine and derivatives:
   6-carboxy-X-rhodamine (ROX)
   6-carboxyrhodamine (R6G)
   lissamine rhodamine B sulfonyl chloride
   rhodamine (Rhod)
   rhodamine B
   rhodamine 123
   rhodamine green
   rhodamine X isothiocyanate
   sulforhodamine B
   sulforhodamine 101
   sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
terbium chelate derivatives

Other fluorescent nucleotide analogs can be used, see, e.g., Jameson, Meth. Enzymol. 278:363-390, 1997; Zhu, Nucl. Acids Res. 22:3418-3422, 1994. U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e. collisional) quenching or by Förster or fluorescence resonance energy transfer (“FRET”). Quenching by FRET is generally used in TaqMan® probes while proximal quenching is used in molecular beacon and scorpion type probes.

In proximal quenching (a.k.a. “contact” or “collisional” quenching), the donor is in close proximity to the quencher moiety such that energy of the donor is transferred to the quencher, which dissipates the energy as heat as opposed to a fluorescence emission. In FRET quenching, the donor fluorophore transfers its energy to a quencher which releases the energy as fluorescence at a higher wavelength. Proximal quenching requires very close positioning of the donor and quencher moiety, while FRET quenching, also distance related, occurs over a greater distance (generally 1-10 nm, the energy transfer depending on R⁻⁶, where R is the distance between the donor and the acceptor). Thus, when FRET quenching is involved, the quenching moiety is an acceptor fluorophore that has an excitation frequency spectrum that overlaps with the donor emission frequency spectrum. When quenching by FRET is employed, the assay may detect an increase in donor fluorophore fluorescence resulting from increased distance between the donor and the quencher (acceptor fluorophore) or a decrease in acceptor fluorophore emission resulting from increased distance between the donor and the quencher (acceptor fluorophore).

The detectable label can be incorporated into, associated with or conjugated to a nucleic acid. Label can be attached by spacer arms of various lengths to reduce potential steric hindrance or impact on other useful or desired properties. See, e.g., Mansfield, Mol. Cell. Probes 9:145-156, 1995.

Detectable labels can be incorporated into nucleic acids by covalent or non-covalent means, e.g., by transcription, such as by random-primer labeling using Klenow polymerase, or nick translation, or, amplification, or equivalent as is known in the art. For example, a nucleotide base is conjugated to a detectable moiety, such as a fluorescent dye, e.g., Cy3™ or Cy5,™ and then incorporated into genomic nucleic acids during nucleic acid synthesis or amplification. Nucleic acids can thereby be labeled when synthesized using Cy3™ or Cy5™-dCTP conjugates mixed with unlabeled dCTP.

Nucleic acid probes can be labeled by using PCR or nick translation in the presence of labeled precursor nucleotides, for example, modified nucleotides synthesized by coupling allylamine-dUTP to the succinimidyl-ester derivatives of the fluorescent dyes or haptens (such as biotin or digoxigenin) can be used; this method allows custom preparation of most common fluorescent nucleotides, see, e.g., Henegariu, Nat. Biotechnol. 18:345-348, 2000.

Nucleic acid probes may be labeled by non-covalent means known in the art. For example, Kreatech Biotechnology's Universal Linkage System® (ULS®) provides a non-enzymatic labeling technology, wherein a platinum group forms a co-ordinative bond with DNA, RNA or nucleotides by binding to the N7 position of guanosine. This technology may also be used to label proteins by binding to nitrogen and sulphur containing side chains of amino acids. See, e.g., U.S. Pat. Nos. 5,580,990; 5,714,327; and 5,985,566; and European Patent No. 0539466.

Labeling with a detectable label also can include a nucleic acid attached to another biological molecule, such as a nucleic acid, e.g., an oligonucleotide, or a nucleic acid in the form of a stem-loop structure as a “molecular beacon” or an “aptamer beacon”. Molecular beacons as detectable moieties are well known in the art; for example, Sokol (Proc. Natl. Acad. Sci. USA 95:11538-11543, 1998) synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (Biochemistry 40:9387-9395, 2001), describing a molecular beacon comprised of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi, Anal. Biochem. 294:126-131, 2001; Poddar, Mol. Cell. Probes 15:161-167, 2001; Kaboev, Nucl. Acids Res. 28:E94, 2000. Aptamer beacons can adopt two or more conformations, one of which allows ligand binding. A fluorescence-quenching pair is used to report changes in conformation induced by ligand binding. See also, e.g., Yamamoto, Genes Cells 5:389-396, 2000; Smimov, Biochemistry 39:1462-1468, 2000.

The nucleic acid probe may be indirectly detectably labeled via a peptide. A peptide can be made detectable by incorporating predetermined polypeptide epitopes recognized by a secondary reporter (e.g., leucine zipper pair sequences, binding sites for secondary antibodies, transcriptional activator polypeptide, metal binding domains, epitope tags). A label may also be attached via a second peptide that interacts with the first peptide (e.g., S-S association).

In certain embodiments, isolated or purified molecules may be preferred. As used herein, the terms “isolated”, “purified” or “substantially purified” refer to molecules, either nucleic acid or amino acid sequences, that are removed from their natural environment, isolated or separated, and are at least 60% free, preferably 75% free, and most preferably 90% free from other components with which they are naturally associated. An isolated molecule is therefore a substantially purified molecule.

Hybridization

The methods of the present invention can incorporate all known methods and means and variations thereof for carrying out DNA hybridization, see, e.g., Sambrook, et al., 1989, Molecular Cloning: A Laboratory Manual, Second Edition, Cold Spring Harbor Press, Plainview, N.Y.

In some applications may be helpful to block the hybridization capacity of repetitive sequences. A number of methods for removing and/or disabling the hybridization capacity of repetitive sequences are known (see, e.g., WO 93/18186). For instance, bulk procedures can be used. In many genomes, including the human genome, a major portion of shared repetitive DNA is contained within a few families of highly repeated sequences such as Alu. These methods exploit the fact that hybridization rate of complementary sequences increases as their concentration increases. Thus, repetitive sequences, which are generally present at high concentration will become double stranded more rapidly than others following denaturation and incubation under hybridization conditions. The double stranded nucleic acids are then removed and the remainder used in hybridizations. Methods of separating single from double stranded sequences include using hydroxyapatite or immobilized complementary nucleic acids attached to a solid support, and the like. Alternatively, the partially hybridized mixture can be used and the double stranded sequences will be unable to hybridize to the probe.

For example, Cot-1 DNA can be used to selectively inhibit hybridization of repetitive sequences in a sample. To prepare Cot-1 DNA, DNA is extracted, sheared, denatured and renatured. Because highly repetitive sequences reanneal more quickly, the resulting hybrids are highly enriched for these sequences. The remaining single stranded (i.e., single copy sequences) is digested with S1 nuclease and the double stranded Cot-1 DNA is purified and used to block hybridization of repetitive sequences in a sample. Although Cot-1 DNA can be prepared as described above, it is also commercially available (BRL).

Hybridization conditions for nucleic acids in the methods of the present invention are well known in the art. For example, hybridization conditions may be high, moderate or low stringency conditions. Ideally, nucleic acids will hybridize only to complementary nucleic acids and will not hybridize to other non-complementary nucleic acids in the sample. The hybridization conditions can be varied to alter the degree of stringency in the hybridization and reduce background signals as is known in the art. For example, if the hybridization conditions are high stringency conditions, a nucleic acid will detectably bind to nucleic acid target sequences with a very high degree of complementarity. Low stringency hybridization conditions will allow for hybridization of sequences with some degree of sequence divergence. The hybridization conditions will vary depending on the biological sample, and the type and sequence of nucleic acids. One skilled in the art will know how to optimize the hybridization conditions to practice the methods of the present invention.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have a high frequency of complementary base sequences.

Exemplary hybridization conditions are as follows. High stringency generally refers to conditions that permit hybridization of only those nucleic acid sequences that form stable hybrids in 0.018M NaCl at 65° C. High stringency conditions can be provided, for example, by hybridization in 50% formamide, 5× Denhardt's solution, 5×SSC (saline sodium citrate) 0.2% SDS (sodium dodecyl sulphate) at 42° C., followed by washing in 0.1×SSC, and 0.1% SDS at 65° C. Moderate stringency refers to conditions equivalent to hybridization in 50% formamide, 5× Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5× Denhardt's solution, 6×SSC, 0.2% SDS, followed by washing in 1×SSC, 0.2% SDS, at 50° C.

As used herein, the terms “complementary” or “complementarity” are used in reference to polynucleotides (i.e., a sequence of nucleotides such as an oligonucleotide or a target nucleic acid) related by the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association”. For example, for the sequence “5′-A-G-T-3′” is complementary to the sequence “3′-T-C-A-5”. Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Complementarity may be “partial” in which only some of the nucleic acids' bases are matched according to the base pairing rules. Or, there may be “complete,” “total,” or “full” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The terms “identity” and “identical” refer to a degree of identity between sequences. There may be partial identity or complete identity. A partially identical sequence is one that is less than 100% identical to another sequence. Preferably, partially identical sequences have an overall identity of at least 70% or at least 75%, more preferably at least 80% or at least 85%, most preferably at least 90% or at least 95%.

Capture of Nucleic Acid Hybridization Complexes

Many methods for immobilizing capture moieties on a variety of solid surfaces are known in the art. The solid surface may be composed of any of a variety of materials, for example, glass, quartz, silica, paper, plastic, nitrocellulose, nylon, polypropylene, polystyrene, or other polymers. The solid support may be in the form of beads, microparticles, microspheres, plates which are flat or comprise wells, shallow depressions, or grooves, microwell surfaces, slides, chromatography columns, membranes, filters, or microchips. In a preferred embodiment, the solid support is in the form of a bead or microparticle. These beads may be composed of, for example, polystyrene or latex. Beads may be of a similar size or may be of varying size. Beads may be approximately 0.1 μm-10 μm in diameter or may be as large as 50 μm-100 μm in diameter, however, smaller and larger bead sizes are possible.

The desired capture moiety may be covalently bound or noncovalently attached. If covalent bonding between a compound and the surface is desired, the solid surface will usually be polyfunctional or be capable of being polyfunctionalized. Functional groups which may be present on the solid surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

Capture of hybridization complexes or probe may be accomplished through contacting a probe containing one member of a binding pair, either alone or as part of a hybridization complex, with a solid support to which the second member (capture moiety) of the binding pair is bound. Capture may be done in solution or solid support and may be done prior to, subsequent to, or simultaneously with hybridization to the nucleic acids of the test sample and the detectably labeled probe.

In a preferred embodiment, hybridization complexes may be captured on commercially available coated beads or microparticles. For instance, biotin end-labeled nucleic acids can be captured on commercially available streptavidin- or avidin-coated beads. Streptavidin or anti-digoxigenin antibody can also be attached to beads or microparticles by protein-mediated coupling, using, for example, protein A following standard protocols. Biotin or digoxigenin end-labeled nucleic acids can be prepared according to standard techniques. Alternatively, paramagnetic particles, such as ferric oxide particles, with or without avidin coating, can be used.

Detection of Hybridization Complexes

Methods of detection of detectably labeled probes incorporated into captured hybridization complexes are known in the art and vary dependent on the nature of the label. In preferred embodiments the detectable label is a fluorescent dye. Fluorescent dyes are detected through exposure of the label to a photon of energy of one wavelength, supplied by an external source such as an incandescent lamp or laser, causing the fluorophore to be transformed into an excited state. The fluorophore then emits the absorbed energy in a longer wavelength than the excitation wavelength which can be measured as fluorescence by standard instruments containing fluorescence detectors. Exemplary fluorescence instruments include spectrofluorometers and microplate readers, fluorescence microscopes, fluorescence scanners, and flow cytometers.

In addition to labeling nucleic acids with fluorescent dyes, the invention can be practiced using any apparatus or methods to detect detectable labels associated with nucleic acids of a sample, an individual member of the nucleic acids of a sample, or, any apparatus or methods to detect nucleic acids specifically hybridized to each other. Devices and methods for the detection of multiple fluorophores are well known in the art, see, e.g., U.S. Pat. Nos. 5,539,517; 6,049,380; 6,054,279; 6,055,325; and 6,294,331. Any known device or method, or variation thereof, can be used or adapted to practice the methods of the invention, including array reading or “scanning” devices, such as scanning and analyzing multicolor fluorescence images; see, e.g., U.S. Pat. Nos. 6,294,331; 6,261,776; 6,252,664; 6,191,425; 6,143,495; 6,140,044; 6,066,459; 5,943,129; 5,922,617; 5,880,473; 5,846,708; 5,790,727; and, the patents cited in the discussion of arrays, herein. See also published U.S. Patent Application Nos. 20010018514; 20010007747; and published international patent applications Nos. WO0146467 A; WO9960163 A; WO0009650 A; WO0026412 A; WO0042222 A; WO0047600 A; and WO0101144 A.

Charge-coupled devices, or CCDs, are used in microarray scanning systems, including practicing the methods of the invention. Color discrimination can also be based on 3-color CCD video images; these can be performed by measuring hue values. Hue values are introduced to specify colors numerically. Calculation is based on intensities of red, green and blue light (RGB) as recorded by the separate channels of the camera. The formulation used for transforming the RGB values into hue, however, simplifies the data and does not make reference to the true physical properties of light. Alternatively, spectral imaging can be used; it analyzes light as the intensity per wavelength, which is the only quantity by which to describe the color of light correctly. In addition, spectral imaging can provide spatial data, because it contains spectral information for every pixel in the image. Alternatively, a spectral image can be made using brightfield microscopy, see, e.g., U.S. Pat. No. 6,294,331.

In a preferred embodiment, hybridized complexes are detected using flow cytometry. Flow cytometry is a technique well-known in the art. Flow cytometers hydrodynamically focus a liquid suspension of particles (e.g., cells or synthetic microparticles or beads) into an essentially single-file stream of particles such that each particle can be analyzed individually. Flow cytometers are capable of measuring forward and side light scattering which correlates with the size of the particle. Thus, particles of differing sizes may be used in invention methods simultaneously to detect distinct nucleic acid segments. In addition fluorescence at one or more wavelengths can be measured simultaneously. Consequently, particles can be sorted by size and the fluorescence of one or more fluorescent labels probes can be analyzed for each particle. Exemplary flow cytometers include the Becton-Dickenson Immunocytometry Systems FACSCAN. Equivalent flow cytometers can also be used in the invention methods.

As readily recognized by one of skill in the art, detection of the hybridization complex can be achieved through use of a labeled antibody against the label of the second labeled probe. For example, in a preferred embodiment, the second probe is labeled with digoxigenin and is detected with a fluorescent labeled anti-digoxigenin antibody. These antibodies are readily available commercially.

The invention will now be described in greater detail by reference to the following non-limiting examples.

### EXAMPLE 1

### Preparation of Labeled Nucleic Acid Probes

Bacterial artificial chromosomes (BACs) containing the BCR locus (BCR-BAC) and BACs containing the ABL locus (ABL-BAC) were used to generate probes to detect the Philadelphia chromosome translocation. These BACs were purchased commercially (Invitrogen). The BACs were grown and isolated using standard methods.

Biotinylation of BACs

The isolated ABL-BAC was biotinylated using a standard nick translation (NT) protocol. 10 μl of ABL-BAC was mixed with NT enzyme, buffer, and biotin-16-dUTP incubated at 65° C. for 1.5 hours. 0.5 M EDTA was added and the mixture incubated at 65° C. for 10 minutes.

Detection Labeling of BACs

The isolated BCR-BAC DNA was digested in aqueous solution with DNAse I for 10 minutes at 37° C. The digestion reaction was stopped with a 10 minute incubation at 65° C. 1 μg of the digested BCR-BAC was ethanol precipitated out of solution using 1/10 volume 3M NaOAc and 2 volumes 100% ethanol and incubating at −70° C. for 30 minutes. The solution was centrifuged at maximum speed for 30 minutes and the resulting DNA pellet was washed in 70% ethanol and allowed to air dry. The DNA pellet was resuspended in 20 μl labeling buffer (0.5M Tris HCL, 1 mM DTT, 0.1M MgSO4, 0.5 mg/ml BSA) denatured at 95° C. for 5 minutes and snap cooled. 1 μl Alexa Fluor 488 was added to the DNA and the mixture centrifuged. The labeled DNA was ethanol precipitated as above and stored at −70° C. until use.

For detection using a detectably labeled antibody, BCR-BAC DNA was nick translation labeled with digoxigenin using the NT method described above.

### EXAMPLE 2

### Hybridization of Labeled BAC Probes and Genomic DNA

Labeled probes were hybridized to a test sample of genomic DNA. The biotinylated probe and digoxigenin-labeled probe were mixed and centrifuged at maximum speed for 30 minutes. The resulting pellet was resuspended in hybridization buffer (50% Formamide, 10% dextran sulfate, 2×SSC, 40 mM sodium phosphate buffer and 1× Denhardt's Solution), incubated at 37° C. for 30 minutes and denatured at 73° C. for 10 minutes. The probe mixture was then cooled on ice for 5 minutes and incubated for 30 hour at 37° C. Denaturation solution (70% deionized Formamide, 0.2×SSC) was then added to the probe mixture.

Genomic DNA was digested with DpnII for 1 hour at 37° C. The digestion was stopped by heat inactivation at 65° C. for 10 minutes. Digested genomic DNA (1 μg) was denatured in denaturation solution (70% deionized Formamide, 0.2×SSC) by incubation at 73° C. for 7 minutes then incubated on ice for 5 minutes.

The denatured probe mixture and denatured genomic DNA were then combined and incubated at 37° C. overnight.

### EXAMPLE 3

### Capture of Hybridization Complex on Solid Support

Hybridization complexes incorporating a biotin-labeled probe were captured on streptavidin-coated beads. 5 μl of streptavidin beads (Bangs Lab, Fishers, Ind.) were washed once with 100 μl TTL solution (100 mM Tris-HCL; pH 8.0, 0.1% Tween 20; and 1 M LiCl) and resuspended in 20 μl TTL. 5 μl probe-DNA complex was added to the beads and the mixture incubated while shaking at room temperature for 30 minutes to form a bead-DNA complex. The bead complex was then washed three times with 2% BSA in phosphate buffered saline (PBS), resuspended in 4% blocking milk, and washed once with 2% BSA in PBS. The bead complex was then resuspended in FITC-labeled anti-digoxigenin antibody at a dilution of 1:500 and rotated for 30 minutes at room temperature in the dark. The bead complex was then washed once with 2% BSA in PBS using a Sorvall CW-2 Cell washer to wash and pellet the beads.

### EXAMPLE 4

### Detection of Hybridization Complex Using Flow Cytometry

The FITC-labeled anti-digoxigenin antibody was detected as a change in fluorescence per bead as measured on a flow cytometer (FACsCalibur, BD San Jose, Calif.) following the manufacturer's instruction.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

That which is claimed is:

1. A method for detecting a target nucleic acid in a test sample, said method comprising:
forming on a solid support a complex comprising the target nucleic acid, a first nucleic acid probe hybridizing to a first segment of the target nucleic acid, said first nucleic acid probe labeled with a detectable label, and a second nucleic acid probe hybridizing to a second segment of the target nucleic acid, said second nucleic acid probe anchored to the solid support, and detecting said complex by detecting incorporated detectable label, wherein at least one of said first and second nucleic probe is at least 50 nucleotides in length.

2. The method of claim 1, wherein said solid support comprises a first member of a binding pair and said second probe comprises a second member of a binding pair which has binding affinity for said first member of a binding pair, and wherein binding of the first member of the binding pair to the second member of the binding pair anchors the second probe to the support.

3. The method of claim 2, wherein said complex is formed by hybridizing said target nucleic acid to said first nucleic acid probe and to said second nucleic acid probe prior to contacting the complex with said solid support comprising a first member of a binding pair.

4. The method of claim 1, wherein said solid support is one or more beads or one or more microwell plates.

5. The method of claim 2, wherein said binding pair is selected from the group consisting of ligand-receptor, a hormone-receptor, an oligonucleotide-complement, and antigen-antibody.

6. The method of claim 5, wherein said ligand-receptor is biotin and streptavidin or avidin.

7. The method of claim 1, wherein said nucleic acid probes are selected from the group consisting of oligonucleotide probes, artificial chromosome probes, fragmented artificial chromosome probes, genomic DNA probes, RNA probes, and recombinant nucleic acid probes.

8. The method of claim 1, wherein the first nucleic acid probe is labeled with a fluorophore.

9. The method of claim 1, wherein said complex is detected on the solid support by flow cytometry.

10. The method of claim 1, wherein said complex is detected by detecting a labeled reagent that binds to the detectable label of the first nucleic acid probe.

11. The method of claim 10, wherein said labeled reagent is a labeled antibody that is specific for the detectable label.

12. A method for detecting the presence or absence of a genetic abnormality in a target nucleic acid in a test sample, said method comprising:
forming on a solid support a complex comprising the target nucleic acid, a first nucleic acid probe hybridizing to a first segment of the target nucleic acid, said first nucleic acid probe labeled with a detectable label, and a second nucleic acid probe hybridizing to a second segment of the target nucleic acid, said second nucleic acid probe anchored to the solid support, and detecting said complex by detecting incorporated detectable label, wherein hybridization of both the first and second probes to the same target nucleic acid indicates detection of genetic abnormality in the target nucleic acid, while hybridization of only one of said probes to the same target nucleic acid indicates the absence of a genetic abnormality in the target nucleic acid.

13. The method of claim 12, wherein said solid support comprises a first member of a binding pair and said second probe comprises a second member of a binding pair which has binding affinity for said first member of a binding pair, and wherein binding of the first member of the binding pair to the second member of the binding pair anchors the second probe to the support.

14. The method of claim 13, wherein said complex is formed by hybridizing said target nucleic acid to said first nucleic acid probe and to said second nucleic acid probe prior to contacting the complex with said solid support.

15. The method of claim 12, wherein said solid support is one or more beads or one or more microwell plates.

16. The method of claim 13, wherein said binding pair is selected from the group consisting of ligand-receptor, a hormone-receptor, an oligonucleotide-complement, and antigen-antibody.

17. The method of claim 16, wherein said ligand-receptor is biotin and streptavidin or avidin.

18. The method of claim 12, wherein said nucleic acid probes are selected from the group consisting of oligonucleotide probes, artificial chromosome probes, fragmented artificial chromosome probes, genomic DNA probes, RNA probes, and recombinant nucleic acid probes.

19. The method of claim 12, wherein the first nucleic acid probe is labeled with a fluorophore.

20. The method of claim 12, wherein said complex is detected on the solid support by flow cytometry.

21. The method of claim 12, wherein said complex is detected by detecting a labeled reagent that binds to the detectable label of the first nucleic acid probe.

22. The method of claim 21, wherein said labeled reagent is a labeled antibody that is specific for the detectable label.

23. A method for analyzing nucleic acid from a sample of an individual to determine if the individual has a duplication or deletion associated with a particular chromosomal segment or gene, comprising,
a) forming on a solid support a complex comprising the nucleic acid associated with the particular chromosomal segment or gene which is obtained from the sample, a first nucleic acid probe hybridizing to a first segment of the nucleic acid associated with the particular chromosomal segment or gene, said first nucleic acid probe labeled with a detectable label, and a second nucleic acid probe hybridizing to a second segment of the nucleic acid associated with the particular chromosomal segment or gene, wherein said second nucleic acid probe is anchored to the solid support, and
b) measuring a test value representing the amount of complex formed with nucleic acid associated with the particular chromosomal segment or gene by detecting the amount of detectable label incorporated into the complex, and
c) comparing the amount measured in step b) to a control value obtained for another particular chromosomal segment or gene, wherein an increase in the test value compared to the control value is indicative of a duplication and a decrease in the test value compared to the control value is indicative of a deletion.

24. The method of claim 23, wherein said solid support comprises a first member of a binding pair and said second probe comprises a second member of a binding pair which has binding affinity for said first member of a binding pair, and wherein binding of the first member of the binding pair to the second member of the binding pair anchors the second probe to the support.

25. The method of claim 23, wherein the test value and control value are determined using the same sample.

26. The method of claim 23, wherein a first ratio is obtained using the test value and the control value, and that this ratio is compared to a similar ratio obtained for a test value and a control value from a nucleic acid which has a wildtype sequence for the particular chromosomal segment or gene, and wherein an increase in the first ratio compared to the second ratio is indicative of a duplication and a decrease in the first ratio compared to the second ratio is indicative of a deletion.

27. The method of claim 23, wherein the control value is obtained by forming on a solid support a second complex comprising the nucleic acid associated with a different particular gene, a third nucleic acid probe hybridizing to a first segment of the nucleic acid associated with the different particular gene, said third nucleic acid probe labeled with a detectable label, and a fourth nucleic acid probe hybridizing to a second segment of the nucleic acid associated with the different particular gene, wherein said fourth nucleic acid probe is anchored to the solid support; and
measuring the amount of second complex formed with nucleic acid associated with the different particular gene by detecting the amount of detectable label incorporated into the complex.

28. The method of claim 27, wherein in the case of said second complex, said solid support comprises a first member of a binding pair and said second probe comprises a second member of a binding pair which has binding affinity for said first member of a binding pair, and wherein binding of the first member of the binding pair to the second member of the binding pair anchors the second probe to the support.

29. The method of claim 27, wherein the test value and control value are determined using the same sample.

30. The method of claim 23, wherein the test value and control value are determined in a single reaction vessel, and wherein said detectable labels of said first nucleic acid probe and said second nucleic acid probe are distinguishable.

31. The method of claim 23, wherein the test value and control value are determined in a separate reaction vessel.

32. The method of claim 28, wherein the binding pair members used to determine the test value are different from the binding pair members used to determine the control value.

33. A method for detecting a chromosomal translocation of a target nucleic acid in a test sample, said method comprising,
forming on a solid support a complex comprising the target nucleic acid, a first nucleic acid probe hybridizing to a region of a first chromosome of the translocation, said first nucleic acid probe labeled with a detectable label, and a second nucleic acid probe hybridizing to a region of a second chromosome of the translocation, wherein said second nucleic acid probe is anchored to the solid support, and
detecting the complex by detecting detectable label incorporated into the complex, wherein said detecting indicates the presence of the chromosomal translocation.

34. The method of claim 33, wherein said solid support comprises a first member of a binding pair and said second probe comprises a second member of a binding pair which has binding affinity for said first member of a binding pair, and wherein binding of the first member of the binding pair to the second member of the binding pair anchors the second probe to the support.

35. The method of claim 33, wherein said wherein said translocation is selected from the group consisting of t(9;22), t(6;11), t(11;16), t(8;21), t(8;14), t(4;14), Inv 16, t(5;12), t(11;14), and t(14;18).

36. The method of claim 33, wherein said first chromosome is chromosome 9 and wherein said second chromosome is chromosome 22.

37. The method of claim 33, wherein said region of the first chromosome comprises the ABL locus and wherein said region of the second chromosome comprises the BCR locus.

38. The method of claim 37, wherein detecting said chromosomal translocation indicates that the individual has chronic myelogenous leukemia (CML).

39. A method of determining diagnosis, predicting response to therapy, detecting minimal residual disease or prognosis of a disease in an individual, said method comprising,
a) forming on a solid support a complex comprising the target nucleic acid from a test sample of the individual, a first nucleic acid probe hybridizing to a first segment of the target nucleic acid, said first nucleic acid probe labeled with a detectable label, and a second nucleic acid probe hybridizing to a second segment of the target nucleic acid, wherein said second nucleic acid probe is anchored to the solid support,
b) measuring the amount of complex formed by detecting the amount of detectable label incorporated into the complex; and
c) comparing the amount of complex formed using target nucleic acid from the test sample to the amount of complex formed using target nucleic acid from a reference sample, wherein a difference in amount of complex formed from the test sample as compared to the reference sample is diagnostic, predicts response to therapy, detects minimal residual disease or is prognostic for said disease.

40. The method of claim 39, wherein said solid support comprises a first member of a binding pair and said second probe comprises a second member of a binding pair which has binding affinity for said first member of a binding pair, and wherein binding of the first member of the binding pair to the second member of the binding pair anchors the second probe to the support.

41. The method of claim 38, wherein said reference sample is taken from a normal individual.

42. The method of claim 38, wherein said amount of complex formed using target nucleic acid from a reference sample is obtained by forming on a solid support a complex comprising the target nucleic acid from said reference sample, a first nucleic acid probe hybridizing to a first segment of the target nucleic acid, said first nucleic acid probe labeled with a detectable label, and a second nucleic acid probe hybridizing to a second segment of the target nucleic acid, wherein said second nucleic acid probe is anchored to the support, and measuring the amount of complex formed by detecting the amount of detectable label incorporated into the complex.

43. A method of monitoring progression of a disease, said method comprising, obtaining a first sample containing a target nucleic acid from an individual having a disease,
a) forming on a first solid support a first complex comprising a target nucleic acid from said first sample, a second nucleic acid probe hybridizing to a first segment of said target nucleic acid, said first nucleic acid probe labeled with a detectable label, and a second nucleic acid probe hybridizing to a second segment of said target nucleic acid, wherein said second nucleic acid probe is anchored to the first support, and detecting said first complex by measuring the amount of detectable label incorporated into said complex,
b) obtaining a second sample containing a target nucleic acid from said individual having a disease, wherein said second sample is obtained after the first sample;
c) forming on a second solid support a second complex comprising a target nucleic acid from said second sample, a first nucleic acid probe hybridizing to a first segment of said target nucleic acid, said first nucleic acid probe labeled with a detectable label, and a second nucleic acid probe hybridizing to a second segment of said target nucleic acid, wherein said second nucleic acid probe is anchored to the second support, and detecting said complex by measuring the amount of detectable label incorporated into said complex,
d) comparing the amount of said first complex formed from the first sample to the amount of second complex formed from the second sample, wherein a difference in the amount of first complex and second complex is related to the progression of the disease.

44. The method of claim 43, wherein said first or second solid support comprises a first member of a binding pair and said second probe comprises a second member of a binding pair which has binding affinity for said first member of a binding pair, and wherein binding of the first member of the binding pair to the second member of the binding pair anchors the second probe to the support.

45. The method of claim 43, wherein a decrease in the amount of the second complex from the second sample relative to the amount of first complex from the first sample indicates a reduction in the progression of the disease.

46. The method of claim 43, wherein said target nucleic acid in said first and second complex contains a mutation associated with cancer.

47. A method of measuring the tumor burden in an individual suspected of having cancer, said method comprising,
a) forming on a solid support a first complex comprising a first target nucleic acid from a body fluid test sample, a first nucleic acid probe hybridizing to a first segment of said first target nucleic acid, said first nucleic acid probe labeled with a detectable label, and a second nucleic acid probe hybridizing to a second segment of said first target nucleic acid, wherein said second nucleic is anchored to the solid support, and detecting said first complex,
b) comparing the amount measured in step a) to a reference value or set of reference values that relate the amount in step a) to tumor burden.

48. The method of claim 47, further comprising,
a) forming on a second solid support a second complex comprising a second target nucleic acid from said test sample, a third nucleic acid probe hybridizing to a first segment of said second target nucleic acid, wherein said third nucleic acid probe is labeled with a detectable label, and a fourth nucleic acid probe hybridizing to a second segment of said second target nucleic acid, wherein said fourth nucleic acid probe is anchored to the second solid support, and detecting said second complex;
b) determining a ratio of the value obtained from the first target nucleic acid to the value obtained from the second target nucleic acid; and
c) comparing the ratio determined in step b) to a reference ratio or set of reference ratios that relate the ratio in step b) to tumor burden.

49. The method of claim 47, wherein said first or second solid support comprises a first member of a binding pair and said second probe comprises a second member of a binding pair which has binding affinity for said first member of a binding pair, and wherein binding of the first member of the binding pair to the second member of the binding pair anchors the second probe to the support.

50. The method of claim 47 wherein said first and second solid supports are one in the same.
